(19) United States
(12) Patent Application Publication
(10) Pub. No.: US 2024/0221920 A1
(43) Pub. Date: Jul. 4, 2024

(54) Smart Hospital Bed Operating System and Method using Big Data

(71) Applicant: Seoul National University Hospital, Seoul (KR)

(72) Inventors: Sung Jae JUNG, Seongnam-si (KR); Joong Seob KIM, Seongnam-si (KR); Sung Ho LEE, Seongnam-si (KR); Dong-gil KIM, Seongnam-si (KR); Chang-ho YUN, Seongnam-si (KR)

(21) Appl. No.: 18/251,949

(22) PCT Filed: Nov. 5, 2021
(86) PCT No.: PCT/KR2021/095093

§ 371 Date: Feb. 12, 2024

(30) Foreign Application Priority Data
Nov. 5, 2020 (KR) 10-2020-0146814

(51) Int. Cl. G16H 40/20; G16H 10/60; G16H 50/50

(52) U.S. Cl. CPC G16H 40/20; G16H 10/60; G16H 50/50

(57) ABSTRACT

Embodiments relate to a smart hospital bed operating method and a smart hospital bed operating system performing same, the method comprising the steps of: receiving a hospital bed assignment query including a hospitalization indication condition of a waiting patient; searching for a hospital bed that matches the hospitalization indication condition; and if a list of searched hospital beds does not include an empty hospital bed, pre-assigning, to the waiting patient, an inpatient bed occupied by an inpatient on the basis of a hospital bed assignment plan table including an expected discharge date of the inpatient.

### BACKGROUND OF THE INVENTION

### Field of the Invention

The present invention relates to a hospital bed operating system, and more specifically, to a smart hospital bed operating system that uses big data to optimize supply of hospital beds according to demand for the same, predict the supply and demand, and perform balanced management.

### Description of Related Art

In reality, most hospitals have an insufficient number of hospital beds compared to the number of patients requiring hospitalization. In particular, in the case of South Korea, the concentration of patients in tertiary hospitals is noticeable due to high trust and vague expectations for tertiary general hospitals.

Therefore, the tertiary general hospitals suffer from hospital bed shortages and frequent delays in hospitalization. This not only directly affects the patients' health, but also interferes with the patients' daily life and causes dissatisfaction with the hospitals.

### PRIOR ART LITERATURE

### Patent Literature

1. Korean Patent Publication No. 10-2018-0003345 (Jan. 9, 2018)

### BRIEF SUMMARY OF THE INVENTION

### Technical Problem

The present invention can provide hospital beds at the right time to patients with severe diseases by predicting and managing the supply and demand for hospital beds, and can implement a smart hospital bed operating system that maximizes hospital bed utilization and provides hospitalization to as many patients as possible.

### Solution to Problem

A smart hospital bed operating method performed by a computing device comprising a processor, according to an aspect of the present invention, may include receiving a hospital bed assignment query including a hospitalization indication condition for a waiting patient, searching for a hospital bed that matches the hospitalization indication condition, and if a searched list of hospital beds does not include an empty hospital bed, pre-assigning a hospital bed occupied by an inpatient to the waiting patient based on a hospital bed assignment plan table including the expected discharge date of the inpatient.

In an embodiment, the method may further include calculating the expected length of stay of the inpatient by applying inpatient information of the inpatient to a pre-modeled prediction function, creating a hospital bed assignment plan table including the calculated expected length of stay, and assigning the hospital bed to the waiting patient based on the hospital bed assignment plan table. The hospital bed assignment plan table consists of inpatient hospital beds, inpatients occupying the inpatient hospital beds, and days.

In an embodiment, the expected length of stay of the inpatient may be calculated by the prediction function consisting of a dependent variable and a plurality of independent variables. The dependent variable includes the expected length of stay from the hospitalization indication date to the expected discharge date of inpatients, and the independent variables include one or more of gender, diagnosis code, surgery code, treatment code, or measurement value describing a patient's condition of the waiting patient.

In an embodiment, coefficients of the independent variables in the prediction function may be determined by an optimization algorithm, and the optimization algorithm may include job shop scheduling, Petri net, or CPU scheduling.

In an embodiment, the hospital bed assignment plan table may include a plurality of cells, each of which includes an empty cell associated with hospital beds and days and a fill cell associated with hospital beds, inpatients, and days. The assigning of the hospital bed to the waiting patient based on the hospital bed assignment plan table may include creating a finite state machine (FSM) of the waiting patient, setting a state of the FSM based on the previous history of the waiting patient of the FSM, where if there is no previous history, the state is set to an initial state, and placing the FSM on the hospital bed assignment plan table according to a code of conduct predefined for each state of the FSM.

In an embodiment, the placing of the FSM on the hospital bed assignment plan table according to the code of conduct predefined for each state of the FSM may include placing the FSM whose state is set to the first-1 state in the earliest empty cell among the empty cells in the hospital bed assignment plan table and updating the state of the FSM to a first-2 state, where the earliest empty cell is an empty cell associated with the inpatient hospital bed with the earliest expected admission date in the hospital bed assignment plan table, and updating the state of the FSM to the second state if there is no event for a first predetermined period of time, The second state indicates that assignment is completed

In an embodiment, the placing of the FSM on the hospital bed assignment plan table according to the code of conduct predefined for each state of the FSM may further include sending a reservation notification message including the fact that assignment has been completed to a waiting patient of the FSM in the second state, The reservation notification message includes hospital bed resource information about a hospital bed associated with the cells in which the FSM is placed.

In an embodiment, if the urgency of admission included in the hospitalization indication condition satisfies a priority condition, the FSM of the waiting patient who satisfies the priority condition may include an attribute of maximum tolerable waiting time, and the placing of the FSM on the hospital bed assignment plan table according to the code of conduct predefined for each state of the FSM may include comparing an expected waiting time for the inpatient hospital bed associated with empty cells in which the FSM in the first-2 state is placed with a preset maximum tolerable waiting time, and if the comparison results in that the expected waiting time is equal to or shorter than the maximum tolerable waiting time, updating the state of the FSM to a second state.

In an embodiment, the placing of the FSM on the hospital bed assignment plan table according to the code of conduct predefined for each state of the FSM may include if the comparison results in that the maximum tolerable waiting time is longer than the expected waiting time for the tentatively-assigned hospital bed, sending a concession request signal to another FSM, and updating the state of the FSM from the first-2 state to a first-3 state, where the other FSM is a recipient FSM and includes another FSM tentatively-assigned to another hospital bed with an expected discharge date that is equal to or earlier than the maximum tolerable waiting time, and upon receiving a concession request acceptance signal from the recipient FSM, replacing the FSM to cells where the recipient FSM that transmitted the concession request acceptance signal is placed, and updating the replaced FSM to the second state.

In an embodiment, the method may further include when the recipient FSM has the first-2 state and receives a concession request signal from a requester FSM, comparing the urgency of admission of the recipient FSM with the urgency of admission of the requester FSM, when the urgency of admission of the requester FSM is higher than that of the recipient FSM as a result of the comparison, transmitting a concession request acceptance signal to the requester FSM, and when the urgency of admission of the requester FSM is equal to or lower than that of the recipient FSM, transmitting a concession request rejection signal to the requester FSM.

A computer-readable recording medium according to another aspect of the present invention may record a program for performing the smart hospital bed operating method according to the above-described embodiments.

A hospital bed operating system using big data, according to another aspect of the present invention may include a hospital bed management unit configured to obtain hospital bed resource information, inpatient information, and waiting patient information, a hospital bed assignment unit configured to search for a hospital bed matching a hospitalization indication condition upon receiving a hospital bed assignment query including the hospitalization indication condition, and pre-assign a hospital bed occupied by an inpatient to a waiting patient based on a hospital bed assignment plan table including the inpatient's expected discharge date when the searched list of hospital beds does not include an empty hospital bed, and a hospital bed schedule prediction unit configured to calculate the expected length of stay of the inpatient by applying inpatient information of the inpatient to a pre-modeled prediction function.

In an embodiment, the hospital bed operating system may further include a database storing the hospital bed resource information, the inpatient information, and the waiting patient information.

In an embodiment, the hospital bed operating system may further include a user interface provision unit that provides the hospital bed assignment plan table to a user.

### Advantageous Effects of the Invention

A smart hospital bed operating system according to one aspect of the present invention may enable timely hospitalization for patients who need hospitalization by utilizing limited hospital bed resources as efficiently as possible in situations where hospital beds are insufficient, may provide hospitalization to as many patients as possible, and may provide a confirmed hospitalization schedule. Accordingly, the smart hospital bed operating system may reduce patients' and guardians' anxiety about treatment delays and help them lead their daily lives, thereby improving customer satisfaction, including patients and guardians, and even improving hospital profits.

The effects of the present invention are not limited to the effects mentioned above, and other effects not mentioned will be clearly understood by those skilled in the art from the description of the claims.

### BRIEF DESCRIPTION OF THE SEVERAL VIEWS OF THE DRAWINGS

FIG. 1 is a conceptual diagram of a smart hospital bed operating system, according to an embodiment of the present invention.

FIG. 2 is a conceptual diagram of the operation of a smart hospital bed operating system 1, according to an embodiment of the present invention.

FIG. 3 is a diagram of a hospital bed assignment plan table, according to an embodiment of the present invention.

FIG. 4 is a diagram of a hospital bed assignment plan table further indicating the expected hospitalization period for waiting patients, according to an embodiment of the present invention.

FIG. 5 is a diagram of a hospital bed assignment plan table, according to an embodiment of the present invention.

FIG. 6 is a flowchart of a smart hospital bed operating method, according to an embodiment of the present invention.

FIG. 7 is a flowchart of the operation of assigning a hospital bed to an FSM according to a code of conduct, according to an embodiment of the present invention.

FIG. 8 is a flow diagram of the operation of another FSM receiving a concession request according to a code of conduct, according to an embodiment of the present invention.

### DETAILED DESCRIPTION OF THE INVENTION

### Mode for Invention

The terminology used herein is only intended to refer to specific embodiments and is not intended to limit the invention. As used herein, singular forms include plural forms unless clearly indicated otherwise. As used in the specification, the meaning of “comprising” specifies a particular characteristic, area, integer, step, operation, element and/or ingredient, and does not exclude the presence or addition of another characteristic, area, integer, step, operation, element and/or ingredient.

Although not defined differently, all terms including technical and scientific terms used herein have the same meaning as those generally understood by those skilled in the art in the technical field to which the present invention pertains. Terms defined in commonly used dictionaries are further interpreted as having meanings consistent with related technical literature and currently disclosed content, and are not interpreted in ideal or very formal meanings unless defined.

In this specification, big data refers to a large amount of structured, unstructured or semi-structured data, and may include building-related information, actual transaction price information, and the like. The structured data is data stored in fixed fields, for example, relational databases, spreadsheets, etc. Additionally, the unstructured data is data that is not stored in fixed fields, and includes, for example, text documents, images, videos, and voice data. Additionally, the semi-structured data is data that is not stored in fixed fields but includes metadata or schema, such as XML, HTML, and text.

Hereinafter, embodiments of the present invention will be described in detail with reference to the drawings.

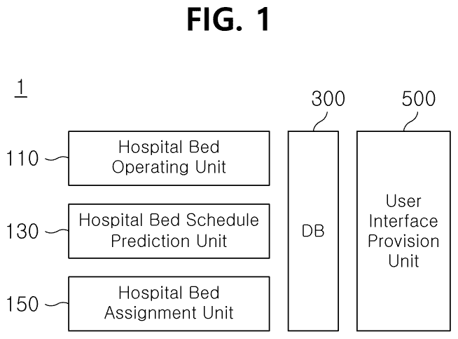

FIG. 1 is a conceptual diagram of a smart hospital bed operating system according to an embodiment of the present invention.

Referring to FIG. 1, a smart hospital bed operating system 1 includes a hospital bed management unit 110, a hospital bed schedule prediction unit 130, and a hospital bed assignment unit 150. In an embodiment, the smart hospital bed operating system 1 may further include a database 300 and/or a user interface provision unit 500.

The smart hospital bed operating system 1 according to embodiments may be entirely hardware, entirely software, or may have aspects that are partly hardware and partly software. For example, a device may collectively refer to hardware equipped with data processing capabilities and operating software for running the same. In this specification, terms such as “unit,” “module,” “device,” or “system” are intended to refer to a combination of hardware and software driven by the hardware. For example, the hardware may be a data processing device including a central processing unit (CPU), a graphics processing unit (GPU), or other processors. Additionally, software may refer to a running process, object, executable file, thread of execution, program, etc.

The database 300 may include a client data store 301, a hospital bed resource data store 310, an inpatient data store 330, a waiting patient data store 350, and/or a training data store 370. It will be clear to those skilled in the art that these data stores are not components of a general database, and that the database 300 may also include other data stores not explicitly mentioned herein.

The database 300 may be implemented using any suitable database management system, such as Microsoft SQL Server, Oracle, SAP, IBM DB2, etc.

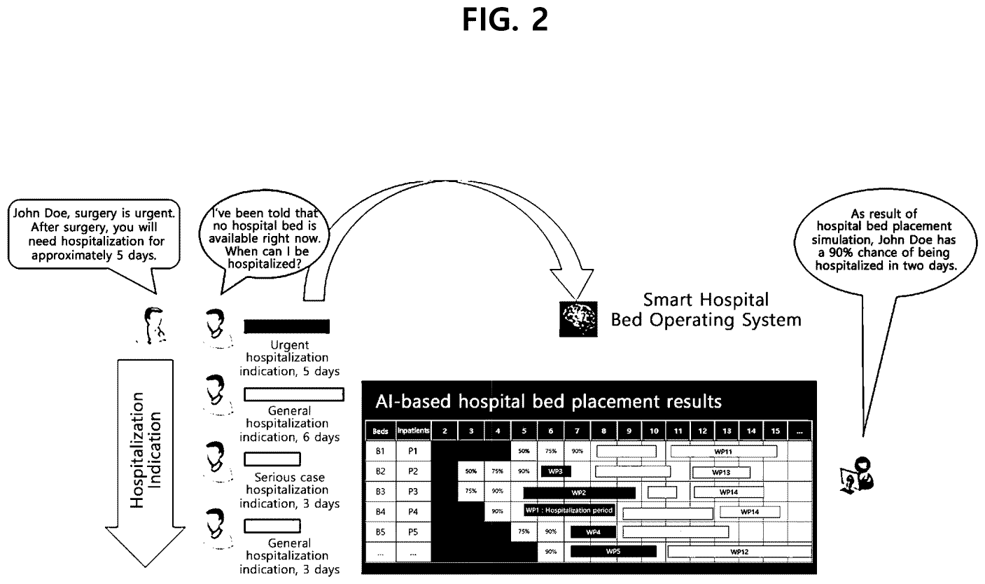

FIG. 2 is a conceptual diagram of the operation of a smart hospital bed operating system 1 according to an embodiment of the present invention.

Referring to FIG. 2, when receiving a request from a waiting patient to assign a hospital bed (i.e., a hospital bed assignment query), the smart hospital bed operating system 1 assigns a hospital bed in the hospital to the waiting patient in response to the hospital bed assignment query. If there is no empty hospital bed in the hospital, the hospital bed assignment query is transmitted to the smart hospital bed operating system 1 through a client module (not shown).

The client module manages the interaction between the client in the hospital and the smart hospital bed operating system 1, includes program code that executes processing logic for the client, related to information that may be requested by other components of the smart hospital bed operating system 1, and is a means of executing the same. Each client is represented by an individual client object in the smart hospital bed operating system 1. Each client object has a unique client ID and a client profile. In certain embodiments, clients may also include medical staff within a hospital.

Referring again to FIG. 1, the hospital bed management unit 110 acquires hospital bed resource information, inpatient information, and waiting patient information. The hospital bed management unit 110 may store the acquired information in the database 300.

The hospital bed resource information is information related to hospital bed resources held by a hospital accessible to the smart hospital bed operating system 1. In certain embodiments, the hospital bed resource information includes one or more of the following: the grade of hospital beds, the number of patients in hospital beds, the gender of patients in hospital beds, the ranking of medical departments that can use the hospital beds, and the characteristics of hospital beds. The number of patients in hospital beds indicates the number of patients in a hospital room including the corresponding hospital beds. The number of patients in hospital beds may be, for example, the number of patients in a single room or a shared room. The gender of patients in hospital beds indicates the gender of the patients who can be admitted to that hospital beds. The characteristics of hospital beds include information on hospital beds designated for each disease. For example, the characteristics of hospital beds include whether the hospital beds can only be used for specific patient groups, such as hospitalization for specific purposes such as maternity or neurophysiological tests.

The inpatient information is information about patients who are hospitalized in hospital beds. In certain embodiments, the inpatient information includes one or more of the gender, age, health information, hospitalization plan, severity, and expected medical expenses of inpatients. The health information includes underlying diseases.

The waiting patient information is information about patients waiting to be hospitalized in hospital beds. The waiting patient information includes individual waiting patient information about each waiting patient, or overall waiting patient information about the entire group of waiting patients waiting for admission. In certain embodiments, the waiting patient information includes one or more of gender, age, morbidity, health information, hospitalization plan, severity, expected medical expenses, and hospital room preference information of waiting patients, and trends of waiting patients in outpatient/emergency rooms. The trends of waiting patients include daily and monthly increase/decrease rates. The hospital room preference information includes information about the number of patients in a hospital room (e.g., single room or shared room).

The hospital bed management unit 110 may determine the urgency of admission of waiting patients based on the waiting patient information. The higher the urgency of admission for waiting patients, the more patients require prompt hospitalization. The hospital bed management unit 110 classifies a number of waiting patients according to the calculated urgency of admission.

In an embodiment, the hospital bed management unit 110 may determine the urgency of admission of waiting patients based on at least one of the severity and the trends of waiting patients in outpatient/emergency rooms. For example, if the severity is high, a first urgency of admission may be determined, if the severity is intermediate, a second urgency of admission may be determined, and if the severity is low, a third urgency of admission may be determined.

In addition, the hospital bed management unit 110 may determine the urgency of admission of waiting patients based on other attributes of waiting patient information other than the severity and the trends of waiting patients in outpatient/emergency rooms. For example, the hospital bed management unit 110 may calculate an urgency score for each waiting patient by weight-summing a plurality of sub-attributes included in the waiting patient information, and may determine the urgency of admission of waiting patients based on the calculated urgency score.

In an embodiment, the hospital bed management unit 110 may set the urgency of admission of waiting patients based on user input. For example, for urgent patients, a user input setting them as a first urgency of admission (e.g., urgent in FIG. 2) may be entered, for severely-ill patients, a user input setting them as a second urgency of admission (e.g., severe case in FIG. 2) may be entered, and for general patients, a user input setting them as a third urgency of admission (e.g., general in FIG. 2) may be entered.

The hospital bed schedule prediction unit 130 predicts the discharge date of inpatients. In an embodiment, the hospital bed schedule prediction unit 130 includes a prediction function that predicts the patients' length of stay. The prediction function is a function generated based on data of previously hospitalized/discharged patients. The previously hospitalized/discharged patients refer to patients who were admitted to the hospital and then discharged.

The prediction function is a function that formalizes the relationship between a number of factors related to the discharge of patients in the past and the actual length of stay in the past. In certain embodiments, the prediction function may be implemented as a regression function. For example, the prediction function may be generated by selecting one or more variables from a number of variables associated with the discharge of patients, and calculating a coefficient of each variable. The selecting of variables is performed based on user specification or correlation with the output. In some embodiments, the correlation with the output may be calculated by various correlation analysis techniques, such as those used in regression analysis.

For example, the variables may include one or more of waiting patients' gender, diagnosis code, surgery code, treatment code, and first to nth measurements describing the patients' condition. The measurements include various bio-signal measurements.

In an embodiment, the prediction function may be expressed as the following equation.

$$\begin{matrix}Y = β0 \\ + {β}_{1} * {X}_{1} ( diagnosis code of patients ) \\ + {β}_{2} * {X}_{2} ( surgery code of patients ) \\ + {β}_{3} * {X}_{3} ( treatment code of patients ) \\ + … \\ + {β}_{n} * {X}_{n} {n}^{t h} ( first to {n}^{th} measurements describing the patients ' condition )\end{matrix} \qquad [ Equation 1 ]$$

Y is a dependent variable, which is the expected length of stay from the hospitalization indication date to the expected discharge date. The expected length of stay is expressed as a relative time based on the expected discharge date and the hospitalization indication date.

X_{N} represents an independent variable (N is an integer), β_{N} represents a regression coefficient (N is an integer), ( ) represents an item corresponding to each variable. N may vary depending on the attributes to be reflected. In Equation 1 above, the error is ignored, but in other embodiments, an error term (ε) may be further added.

In certain embodiments, the coefficients of variables in the prediction function may be determined based on various optimization algorithms. The optimization algorithms may include, for example, job shop scheduling, Petri net, CPU scheduling algorithms, etc., but are not limited thereto. The CPU Scheduling algorithms include Multi Queue Multi Processor Scheduling (MQMS), First Come First Served, FIFO, or Pre-emptive vs. Non Pre-emptive.

The expected length of stay of inpatients may be calculated by applying the inpatient information of the inpatients to the prediction function of Equation 1, whose variables are determined by optimization algorithms.

Additionally, the above-described hospital bed prediction function may be generated by a modeling unit (not shown). The modeling unit may generate the prediction function based on training data stored in the database 300. The training data is data of previously hospitalized/discharged patients, and consists of, for example, a combination of attribute information corresponding to independent variables forming the prediction function and the patients' actual length of stay. The hospital bed schedule prediction unit 130 may calculate the expected discharge date of inpatients using the prediction function generated by the modeling unit.

In other embodiments, the prediction function may be established by establishing each regression function for each medical department, surgery code, or treatment code and constructing a single regression equation integrating each regression function.

The hospital bed schedule prediction unit 130 may supply the calculation result of the expected length of stay of inpatients to the hospital bed management unit 110. Then, the hospital bed management unit 110 may store the calculation result as the inpatient information of inpatients in the database 300.

The hospital bed schedule prediction unit 130 supplies the expected length of stay of inpatients to the hospital bed assignment unit 150. The hospital bed assignment unit 150 manages the discharge schedule of inpatients in the hospital and the admission schedule of waiting patients based on the expected length of stay from the hospital bed schedule prediction unit 130.

Additionally, multiple hospitalization indications are assumed to be assigned to specific hospital beds occupied by inpatients. Multiple patients waiting for specific hospital beds are assigned sequentially based on the discharge date of the previous inpatients. If the hospital bed schedule prediction unit 130 calculates the expected discharge date and expected length of stay of the inpatients using the above-mentioned Equation 1, a first waiting patient may be treated as a new inpatient based on the expected discharge date of the current inpatients. Then, the patient information of the first waiting patient may be applied to Equation 1 above as patient information of the new inpatient to calculate the expected discharge date and expected length of stay of the first waiting patient. For example, the expected admission timing for the next waiting patient may be calculated by adding the expected length of stay to the expected discharge date of the first waiting patient, who is treated as a new inpatient.

Through this process, the waiting period of other waiting patients subsequent to the first waiting patient is calculated, and the calculation result is also supplied to the hospital bed management unit 110 and/or the hospital bed assignment unit 150.

The hospital bed assignment unit 150 responds to the hospital bed assignment query and determines the hospital bed to be assigned to the waiting patient in the query. When the smart hospital bed operating system 1 receives a hospital bed assignment query, the hospital bed assignment unit 150 may assign a hospital bed to a waiting patient responding to the hospital bed assignment query and/or determine a schedule for the waiting patient to be admitted.

The hospital bed assignment unit 150 may calculate the inpatient's expected discharge date or the waiting patient's expected admission date based on the expected length of stay of the inpatient. The expected discharge date or expected admission date may be calculated in an absolute date format (e.g., year, month, day).

The hospital bed assignment query includes identification information of the waiting patient requesting hospital bed assignment. The identification information may include, for example, a hospital identification code, resident registration number, driver's license number, phone number, address, etc.

In an embodiment, the hospital bed assignment query may be entered simultaneously with the input of waiting patient information. For example, when the waiting patient information is entered, hospital bed assignment may be performed automatically.

In the above embodiment, the hospital bed assignment query includes a hospitalization indication condition for the waiting patient. The hospitalization indication condition is a condition regarding hospital beds to be assigned to waiting patients, and may include some or all of waiting patient information and/or hospital bed resource information.

In an embodiment, the hospitalization indication condition may include one or more of the following: the waiting patients' urgency of admission, waiting patients' health information (e.g., disease), the gender of patients in hospital beds, the number of patients in hospital beds, and characteristics of hospital beds. For example, the hospitalization indication condition may include one or more of the following: the urgency of admission, the gender classification, whether the room is a single room, a double room, or a multi-hospital bed room, whether the room is an intensive care unit, whether the hospital bed is an isolated hospital bed, and the medical department.

In addition, the hospitalization indication condition may further include the expected hospitalization period for waiting patients. In an embodiment, the expected hospitalization period for waiting patients may be calculated by the prediction function. The value of the expected length of stay calculated by the prediction function is used as the expected hospitalization period for a waiting patient. In another embodiment, a period set by user input may be used as a hospitalization period for a waiting patient.

In another embodiment, when the smart hospital bed operating system 1 receives a hospital bed assignment query, the hospital bed assignment unit 150 may search the database 300 for the waiting patient information of the waiting patient who entered the hospital bed assignment query, based on the identification information of the waiting patient in the hospital bed assignment query. Due to the search, a hospitalization indication condition for the waiting patient may be obtained.

When the smart hospital bed operating system 1 receives a hospital bed assignment query, the hospital bed assignment unit 150 may search for hospital beds in the hospital that match the hospitalization indication condition. If there is an empty hospital bed that is not occupied by an inpatient among the hospital beds that match the hospitalization indication condition, the hospital bed assignment unit 150 assigns the searched empty hospital bed to the waiting patient in the hospital bed assignment query.

In addition, if all the hospital beds (i.e., inpatient hospital beds) matching the hospitalization indication condition are occupied by inpatients, the hospital bed assignment unit 150 assigns a hospital bed to a waiting patient in the hospital bed assignment query based on the expected discharge date of the waiting patient. In this way, although all hospital beds are occupied by inpatients, hospital beds that are unavailable to waiting patients at the time of request may be assigned to the waiting patients in advance before the inpatients are discharged.

In an embodiment, the hospital bed assignment unit 150 may assign a hospital bed to a waiting patient based on a hospital bed assignment plan table. The hospital bed assignment unit 150 may generate a hospital bed assignment plan table based on the expected length of stay of inpatients and associate the days in the hospital bed assignment plan table and the hospital beds with the waiting patients such that the waiting patients may be expected to be admitted to the associated hospital beds on the associated days.

When the smart hospital bed operating system 1 receives a hospital bed assignment query, the hospital bed schedule prediction unit 130 calculates the expected length of stay of inpatients from the hospitalization indication date. The expected length of stay is calculated for the group of inpatients occupying hospital beds that match the hospitalization indication condition of the hospital bed assignment query. The smart hospital bed operating system 1 causes the hospital bed assignment unit 150 to assign hospital beds to waiting patients who respond to the hospital bed assignment query based on the expected length of stay of inpatients.

The hospital bed assignment unit 150 may also generate a hospital bed assignment plan table. The hospital bed assignment plan table consists of the hospital beds occupied by the inpatients, the inpatients occupying the hospital beds, and the days. The information on hospital beds and inpatients may be displayed on the hospital bed assignment plan table.

Additionally, the hospital bed assignment plan table includes the expected length of stay of inpatients. In certain embodiments, days in the hospital bed assignment plan table may be expressed as a relative time expressed based on the hospitalization indication date, but are not limited thereto. The days may also be expressed as an absolute time.

The hospital bed assignment plan table includes a plurality of cells. Each of the plurality of cells is associated with a hospital bed and a day. The cells that are not associated with inpatients are referred to as empty cells. The empty cells refer to a case where the hospital beds associated with the empty cells are available to waiting patients.

On the other hand, if the hospital beds are inpatient hospital beds, the cells are more associated with inpatients. The cells associated with inpatients are referred to as filled cells. Some or all of the filled cells representing the expected length of stay may refer to a case where the associated hospital beds are not available for waiting patients on the associated days. In an embodiment, if a waiting patient is available for admission on the discharge date of an inpatient, the waiting patient may be placed in a filled cell on the end date of the expected length of stay. Hereinafter, for clarity of explanation, the hospital bed assignment plan table will be described in more detail using embodiments in which a waiting patient is hospitalized the day after the inpatient is discharged.

The hospital bed assignment plan table may be provided to a user by the user interface provision unit 500.

Additionally, the hospital bed assignment unit 150 may calculate the daily discharge probability of inpatients based on the expected length of stay calculated by the prediction function. The hospital bed assignment unit 150 may calculate the discharge probability on the end date of the expected length of stay (i.e., the expected discharge date) as 100%, and may calculate the discharge probability on previous days thereof as lower than 100%. The hospital bed assignment unit 150 may determine that there is a possibility of discharge if the daily discharge probability is greater than or equal to a preset threshold probability.

The daily discharge probability is a probability that an inpatient will actually be discharged on that day. The threshold probability which is a level of probability that the inpatient is not discharged may be, for example, 50%, but is not limited thereto.

In an embodiment, the smart hospital bed operating system 1 may classify the daily discharge probability into sections and display the same on the hospital bed assignment plan table so that each section can be identified. For example, a mark for each section (e.g., text, color, brightness, etc.) may be assigned in advance. The daily discharge probability for that day is displayed on the hospital bed assignment plan table with a mark corresponding to the grade to which the calculated daily discharge probability belongs.

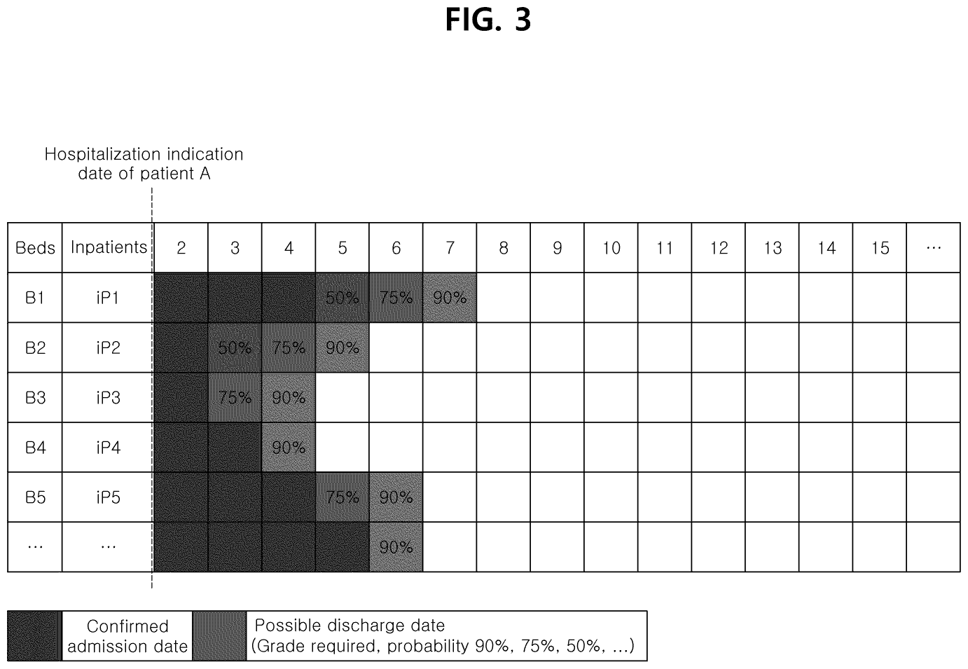

FIG. 3 is a diagram of a hospital bed assignment plan table according to an embodiment of the present invention.

Referring to FIG. 3, the hospital bed assignment plan table may include a list of inpatient hospital beds (B1, B2, B3, . . . in FIG. 3) matching the hospitalization indication condition and a list of inpatients (iP1, iP2, iP3, . . . in FIG. 3) occupying the inpatient hospital beds.

The hospital bed assignment plan table may also include the expected length of stay for each patient. For example, if the expected length of stay of an inpatient B1 is calculated to be 7 days, the hospital bed assignment plan table shows a period indicating that the expected length of stay is 7 days.

The expected length of stay of the inpatient may be expressed in cell form as shown in FIG. 3, but is not limited thereto. For example, the expected length of stay of the inpatient may be expressed in bar form.

If the daily discharge probability is classified into a range of 0-50% (i.e., less than a critical probability range), a range of 50-75%, a range of 75-90%, and a range of 90-100%, each range is assigned a different color. As shown in FIG. 3, the closer the prediction function is to the end date of the expected length of stay, the higher the probability of discharge.

In this way, the smart hospital bed operating system 1 may visually express the expected length of stay of inpatients on the hospital bed assignment plan table. For days within the expected length of stay that have daily discharge probabilities greater than the threshold probability, the daily discharge probabilities may be further expressed so as to be distinct from each other.

The hospital bed assignment unit 150 places waiting patients in a hospital bed assignment plan table that indicates the expected length of stay of the inpatients, and assigns the inpatient hospital beds to the waiting patients. When the hospital bed assignment unit 150 places a waiting patient in empty cells (or space of empty cells) in the hospital bed assignment plan table, the hospital bed associated with the cells where the waiting patient is placed may be assigned to the waiting patient on that date.

For example, in FIG. 3, a series of cells associated with a hospital bed B2 includes cells after the 6th day from the hospitalization indication date for patient A as empty cells. If a waiting patient is placed in a cell on the 6th day or later, the hospital bed B2 may be assigned to the waiting patient in the schedule of the cell where the waiting patient is placed.

In an embodiment, the hospital bed assignment unit 150 may form a finite state machine (FSM) corresponding to the hospitalization indication of the hospital bed assignment query. The hospital bed assignment unit 150 regards the hospitalization indication of the hospital bed assignment query as an object such as a robot that operates on its own. The object, which is the hospitalization indication, may also be referred to as FSM. The hospital bed assignment unit 150 places the FSM on the hospital bed assignment plan table and assigns a hospital bed to a waiting patient of the FSM.

The FSM has the following characteristics: 1) “states” of the FSM are limited; 2) only “one” state at a time is possible; 3) “input” or “event” is transferred to the FSM; and 4) each state can “transition” to the next state depending on the input.

The FSM formed in response to the hospital bed assignment query has condition attributes that describe the hospitalization indication conditions and state attributes that describe the states of the FSM.

In the above example, if the hospitalization indication conditions include one or more of the following: the urgency of admission, the gender classification, whether the room is a single room, a double room, or a multi-hospital bed room, whether the room is an intensive care unit, whether the hospital bed is an isolated hospital bed, and the medical department, the FSM has condition properties corresponding to each hospitalization indication condition.

In some embodiments, if the urgency of admission of the FSM satisfies a preset priority condition, the FSM is created to further have the attribute of maximum tolerable wait time. Additionally, the FSM may further have the attribute of number of concessions allowed.

The preset priority condition refers to whether the urgency of admission included in the hospitalization indication condition belongs to a patient with a relatively high priority. In certain embodiments, the priority condition refers to a case indicating an urgent or severely ill patient. For example, when the FSM of an urgent patient is created S451, step S454 is performed.

The state of FSM indicates whether a hospital bed has been assigned to a waiting patient subject to hospitalization indication.

In an embodiment, the state of each FSM may include a first state (“searching for hospital beds”) and a second state (“assignment completed”). In some embodiments, the state of each FSM may further include a third state (“patient notification completed”) and a fourth state (“patient in hospital”). In addition, the first state may include a first-1 state (“searching for empty hospital bed”), a first-2 state (“tentative assignment”), a first-3 state (“concession request sent”), and/or a first-4 state (“concession request received”). The first state represents a state in which the hospital bed has not been assigned to the FSM. The first-2 state is a state in which the FSM of a waiting patient has been placed in empty cells on the hospital bed assignment plan table, but the hospital bed assignment has not yet been confirmed, and represents a tentative assignment state. The first-2 state is distinguished from the second state in which the hospital bed has been assigned to the waiting patient.

The hospital bed assignment unit 150 may specify the state of the FSM of the hospital bed assignment query based on the transfer details of the waiting patient in the hospital bed assignment query. The above transfer details refer to details of a request for hospital bed assignment by a waiting patient.

The hospital bed assignment unit 150 sets the state of the FSM to the initial state when there is no previous history of the waiting patient in the hospital bed assignment query. If previous history exists, the state of the FSM is set to be determined in the previous history. Depending on the previous history, the FSM may be directly assigned to the second state, third state, or fourth state. For example, if the state of the previous history is the second state, the created FSM is assigned to the second state. For the FSM in the second state, steps S458 to S459 may be performed after being updated to the second state. In certain embodiments, the initial state may be the first state.

The FSM checks its state and operates for the assignment of hospital bed according to the predefined code of conduct for each state.

In addition, a plurality of FSMs may be preferentially assigned or concede their already assigned hospital beds based on their state attributes and/or the condition attributes of waiting patients.

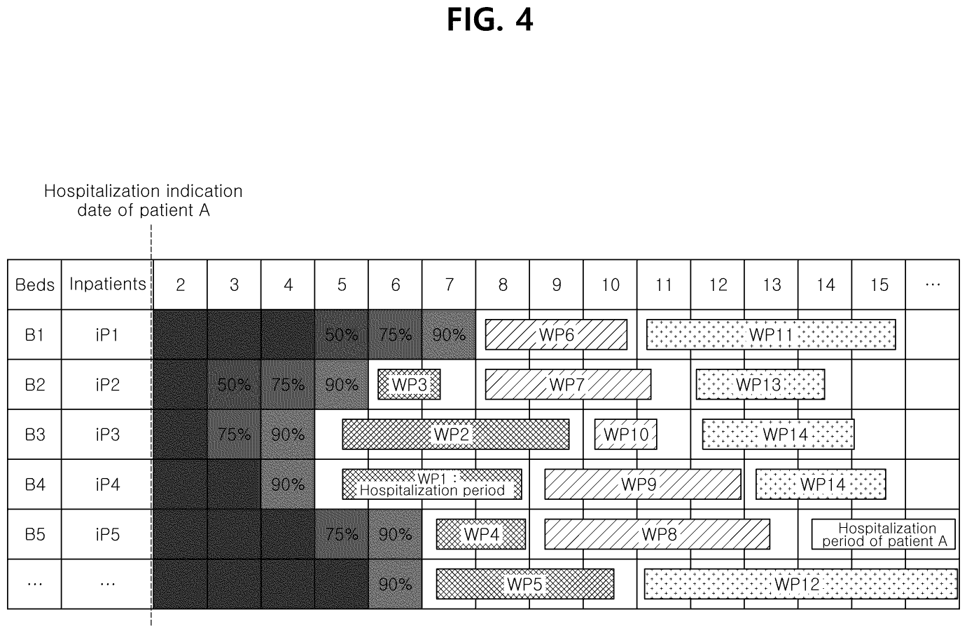

FIG. 4 is a diagram of a hospital bed assignment plan table further indicating the expected hospitalization period for waiting patients, according to an embodiment of the present invention.

In FIG. 4, before inputting the hospital bed assignment query for patient A, hospital beds have been assigned to other waiting patients (WP1, . . . , WP14).

When the hospital bed assignment query for patient A is received, an FSM for patient A is created, and the FSM for patient A is placed in empty cells on the hospital bed assignment plan table. Then, patient A may be expected to be admitted to a hospital bed of the empty cells for days of the empty cells where patient A is placed. In FIG. 4, patient A may be expected to be admitted to a hospital bed B5 on the 14th day from the hospitalization indication date.

The user interface provision unit 500 may provide the hospital bed assignment plan table of FIG. 3 or FIG. 4 to the user.

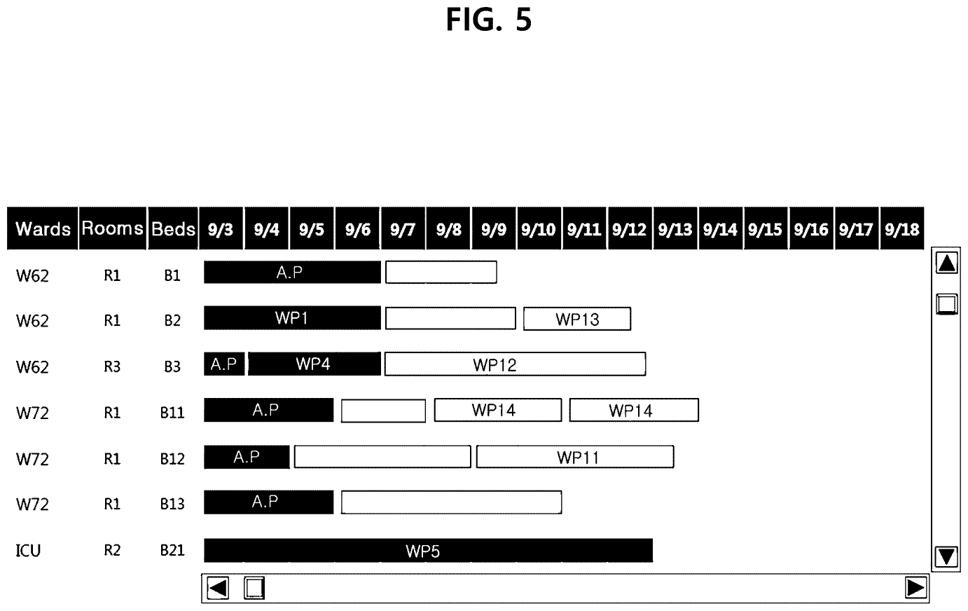

FIG. 5 is a diagram of a hospital bed assignment plan table according to an embodiment of the present invention.

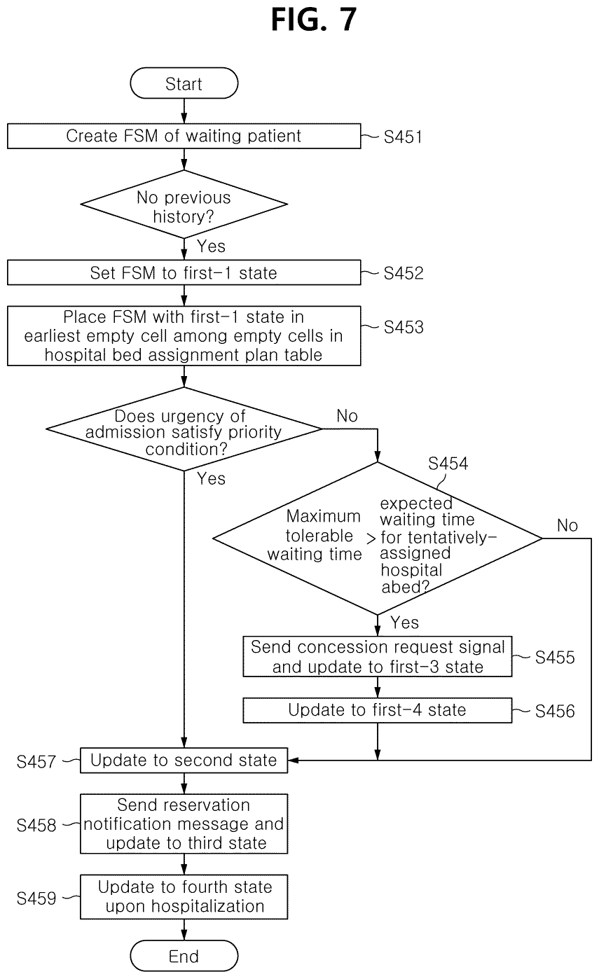

Referring to FIG. 5, the hospital bed assignment plan table may be configured to display at least some of the attributes of waiting patients and/or inpatients. For example, the user interface provision unit 500 may subgroup inpatients and/or waiting patients according to severity and state attributes of the FSM, which will be described with reference to FIG. 7 below, and may configure the hospital bed assignment plan table so that the attributes corresponding to each subgroup are identified.

In one example, the attributes (e.g., state attributes) may be identified by color. Then, as shown in FIG. 5, admitted patients are displayed in the same color. However, means of identifying the attributes is not limited to color, and the attributes may be identified through various means.

Additionally, the hospital bed assignment plan table may be configured to move a screen area through scrolling.

The smart hospital bed operating system 1 may provide a hospital bed reservation notification message to a waiting patient for whom hospital bed assignment has been completed. The message includes the expected admission date. In some embodiments, the message may further include a probability of admission on the expected admission date. The expected admission date is calculated based on the days of the hospital bed assignment plan table calculated based on the hospitalization indication date. The probability of discharge of an inpatient is used as the probability of admission.

In addition, if more hospital bed assignment queries are received for waiting patients other than patient A, different inpatient hospital beds may be assigned to other waiting patients.

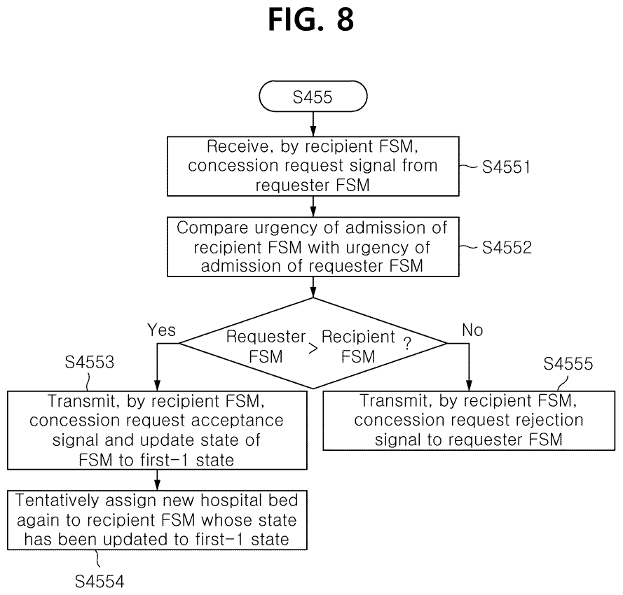

The bed assignment through the FSM is described in more detail with reference to FIGS. 7 and 8 below.

It will be clear to those skilled in the art that the smart hospital bed operating system 1 may include other components not described herein. For example, the smart hospital bed operating system 1 may include other hardware elements necessary for the operations described herein, including a network interface, input devices for data entry, and output devices for display, printing or other data presentation.

The smart hospital bed operating method using big data according to another aspect of the present invention may be performed by a computing device including a processor (for example, the smart hospital bed operating system 1 of FIG. 1). Hereinafter, for clarity of explanation, the present invention will be described in more detail based on embodiments performed by the smart hospital bed operating system 1 of FIG. 1.

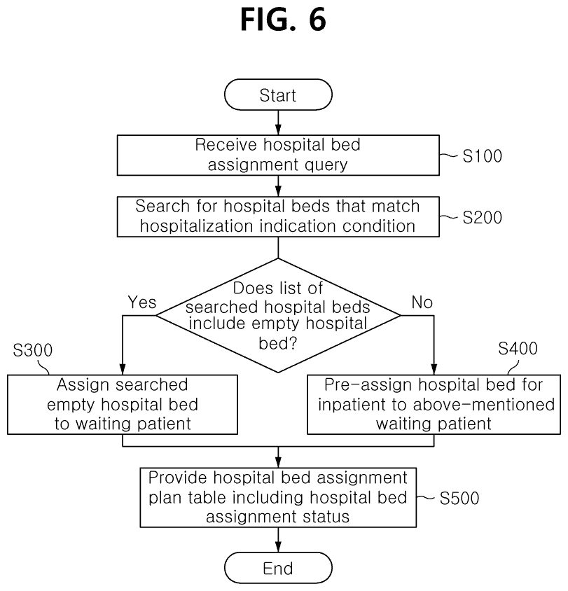

FIG. 6 is a flowchart of a smart hospital bed operating method according to an embodiment of the present invention.

Referring to FIG. 6, the smart hospital bed operating method includes receiving a hospital bed assignment query including identification information of a waiting patient (S100). In certain embodiments, the hospital bed assignment query may be received from a client module.

In some embodiments, the hospital bed assignment query may further include a hospitalization indication condition.

The hospitalization indication condition may include, for example, one or more of the following: urgency of admission, gender classification, whether the room is a single room, a double room, or a multi-hospital bed room, whether the room is an intensive care unit, whether the hospital bed is an isolated hospital bed, and a medical department.

In other embodiments, the hospitalization indication condition may be pre-stored in the database 300 and may be searched based on the identification information of the waiting patient.

Additionally, the smart hospital bed operating method includes searching for a hospital bed that matches the hospitalization indication condition (S200).

In addition, the smart hospital bed operating method includes assigning the searched empty hospital bed to the waiting patient if the list of hospital beds found in step S200 includes an empty hospital bed (S300). The empty hospital bed is a hospital bed that can be used by a waiting patient in response to a hospital bed assignment query.

If the list of hospital beds retrieved in step S200 does not include an empty hospital bed, the smart hospital bed operating method includes pre-assigning the inpatient's hospital bed to the waiting patient (S400).

In an embodiment, step S400 includes: calculating the expected length of stay of the inpatient S410; generating a hospital bed assignment plan table based on the calculated expected length of stay S430; and assigning a hospital bed to the waiting patient based on the hospital bed assignment plan table S450.

The inpatient whose expected length of stay is calculated in step S410 is included in a group of inpatients whose hospitalization indication condition matches, retrieved in step S200.

In step S410, the expected length of stay is calculated based on at least one of hospital bed resource information, inpatient information, and waiting patient information. To this end, the hospital bed resource information, the inpatient information, and the waiting patient information may be acquired in advance or stored in advance before step S100.

In an embodiment, the expected length of stay of the matched inpatient is calculated by applying the hospitalization patient information of the inpatient to the prediction function of Equation 1 (S410). Since the process of calculating the expected length of stay and the modeling process of the prediction function in step S410 are similar to the operation of the hospital bed schedule prediction unit 130, detailed descriptions are omitted.

In step S430, a hospital bed assignment plan table is created showing the expected length of stay for each hospital bed based on the hospitalization indication date. Since the process of creating the hospital bed assignment plan table in step S430 is similar to the operation of the hospital bed assignment unit 150, detailed description will be omitted.

FIG. 7 is a flowchart of the operation of assigning a hospital bed to an FSM according to a code of conduct, according to an embodiment of the present invention.

Referring to FIG. 7, step S450 includes generating an FSM of the waiting patient (S451). If the FSM of the waiting patient created in step S451 is placed in empty cells of the hospital bed assignment plan table, the waiting patient of the FSM is assigned to the hospital bed associated with the empty cells where the FSM is placed. The expected admission date of the waiting patient is calculated based on the days associated with the empty cells where the FSM is placed.

The FSM is placed on the hospital bed assignment plan table according to the predefined code of conduct for each FSM state. Once the FSM is placed, the waiting patient of the placed FSM may be expected to be admitted to a hospital bed associated with the cells where the FSM is placed for days associated with the cells where the FSM is placed.

The FSM searching for a hospital bed that has not yet been assigned has a first state. The FSM with the first state is not placed in an empty cell on the hospital bed assignment plan table.

The FSM placed in empty cells on the hospital bed assignment plan table has a second state. In some embodiments, the state attribute of the FSM with the second state may be updated to a third state and/or a fourth state. Additionally, the FSM for which a hospital bed has not yet been assigned may have a first-1 state, a first-2 state, a first-3 state, or a first-4 state.

The state of the FSM created in step S451 is set based on the previous history of the waiting patient of the FSM (S452).

If there is no previous history of the waiting patient, the state of the FSM is set to the initial state (S452). The initial state is the first state. In an embodiment, the initial state may be the first-1 state. The first-1 state represents a state in which the FSM is searching for cells where the FSM will be placed.

The FSM set to the first-1 state in step S452 is placed in an earliest empty cell among the empty cells in the hospital bed assignment plan table (S453). The earliest empty cell is an empty cell associated with the inpatient hospital bed with the earliest expected admission date in the hospital bed assignment plan table. The earliest empty cell may be a cell associated with the same day or the day after the inpatient's expected discharge date.

Additionally, the state of the FSM placed in the earliest empty cell is updated to the first-2 state (S453). In step S453, the FSM of the waiting patient has been placed in cells, but assignment has not yet been completed. The inpatient hospital bed associated with the cells where the FSM with the first-2 state is placed is a tentatively-assigned hospital bed.

After being updated to the first-2 state in step S453, the state of the FSM may be updated to the second state (S457).

In an embodiment, the update from the first-2 state to the second state is performed if there is no event for a first predetermined period of time (S457). The first predetermined period of time may be, for example, several hours, but is not limited thereto.

In addition, if the FSM of the waiting patient is updated to the second state in step S457, step S450 may further include transmitting a reservation notification message including the fact that assignment has been completed to the waiting patient of the FSM in the second state (S458). The reservation notification message includes hospital bed resource information about a hospital bed associated with the cells in which the FSM is placed. In an embodiment, the reservation notification message may further include expected admission date and/or admission probability. The expected admission date is based on the days associated with the cells where the FSM is placed. For example, if the days associated with the cells where the FSM is placed are expressed as a relative date, they may be changed to an absolute date and included in the message.

When the reservation notification message is transmitted, the state of the FSM is updated to the third state (S458).

In an embodiment, step S450 includes: comparing the expected waiting time for the inpatient hospital bed tentatively-assigned to the FSM in step S453 with a preset maximum tolerable waiting time (S454). The inpatient hospital bed tentatively-assigned to the FSM in step S454 is an inpatient hospital bed associated with empty cells in which the FSM with the first-2 state is placed.

Step S454 is performed when the FSM's urgency of admission satisfies a preset priority condition. The preset priority condition refers to a case where the urgency of admission included in the hospitalization indication condition indicates an urgent or severely ill patient. For example, when the FSM of an urgent patient is created S451, step S454 is performed.

In an embodiment, when the urgency of admission included in the hospitalization indication condition satisfies a priority condition, the FSM of the waiting patient satisfying the priority condition includes the attribute of maximum tolerable waiting time.

The expected waiting time for a tentatively-assigned hospital bed is the time from the hospitalization indication date to the expected admission date of the waiting patient for the tentatively-assigned hospital bed. The expected admission date may be the same day or the next day after the expected discharge date of the inpatient occupying the tentatively-assigned hospital bed.

The maximum tolerable waiting time is the maximum amount of time an urgent patient can wait for recovery. For example, if an urgent patient is admitted beyond the maximum tolerable waiting time, the chances of recovery even with treatment may be drastically reduced.

For waiting patients who require a relative admission priority, the smart hospital bed operating system 1 manages the waiting patients' schedule to be assigned with hospital beds preferentially.

If, as a result of the comparison in step S454, the expected waiting time for the tentatively-assigned hospital bed is equal to or shorter than the maximum tolerable waiting time, the state of the FSM is updated to the second state (S457).

On the other hand, if the comparison result in step S454 shows that the maximum tolerable waiting time is longer than the expected waiting time for the tentatively-assigned hospital bed, a concession request signal is transmitted to another FSM (S455). The concession request signal may be transmitted from the FSM to be compared in step S454. The recipient FSM of the concession request signal includes other FSMs tentatively assigned to other hospital beds with the expected discharge date equal to or earlier than the maximum tolerable waiting time.

Additionally, the state of the FSM that transmitted the concession request signal (e.g., the urgent patient's FSM) is updated to the first-3 state (S455). The transmitting of the concession request signal functions as an event that changes the state of the FSM.

In an embodiment, in order to transmit the concession request signal in step S455, another hospital bed having an expected discharge date equal to or earlier than the maximum tolerable waiting time may be searched. If the searched other hospital bed includes a hospital bed tentatively assigned to another FSM, the concession request signal is transmitted to the other tentatively-assigned FSM (S455). In some embodiments, when a plurality of other FSMs are tentatively assigned to a plurality of other searched hospital beds, the FSM may transmit a concession request signal to the other FSMs tentatively assigned to the hospital beds with the earliest expected discharge date (S455).

Subsequently, when an acceptance signal approving the concession request is received from another FSM (i.e., the recipient FSM) that has received the concession request signal, the FSM is replaced to the cells where the recipient FSM that transmitted the concession request acceptance signal is placed (S457). In addition, the state of the replaced FSM is updated to the second state (S457). The receiving of the concession request acceptance signal also functions as an event that changes the state of the FSM.

In an embodiment, before updating the state of the object to the second state (S457), the FSM that transmitted the concession request signal and received the concession request acceptance signal from another FSM may update its state to the first-4 state (S456). The FSM updated to the first-4 state is updated to the second state after a second predetermined period of time (S457). The second predetermined period of time is less than the maximum tolerable waiting time, and may be, for example, several minutes to several tens of minutes.

Subsequently, when the waiting patient who received the message is actually admitted to the assigned empty hospital bed, the state of the FSM is updated to the fourth state (S459). The admission of the waiting patient is input by updating the inpatient information.

FIG. 8 is a flow diagram of the operation of another FSM receiving a concession request according to a code of conduct, according to an embodiment of the present invention.

In FIG. 8, the other FSM already has the attribute of first-2 state before receiving the concession request signal (S453). For clarity of explanation below, the FSM that transmits the concession request signal as a result of performing step S454 will be referred to as the requester FSM, and the other FSM that receives the concession request signal will be referred to as the recipient FSM, and the code of conduct of the recipient FSM will be described in more detail.

The recipient FSM receives a concession request signal from the requester FSM (S4551). Then, the urgency of admission of the recipient FSM is compared with the urgency of admission of the requester FSM (S4552).

As a result of the comparison, if the urgency of admission of the requester FSM has a higher priority, the recipient FSM transmits a concession request acceptance signal (S4553). For example, if the requester FSM has the attribute of urgent admission and the recipient FSM has the attribute of general admission, the recipient FSM may transmit a concession request acceptance signal (S4553). Additionally, after transmitting the concession request acceptance signal, the recipient FSM updates its state from the first-2 state to the first-1 state (S4553). The transmitting of the concession request acceptance signal functions as an event that changes the state of the recipient FSM.

A new hospital bed is tentatively assigned again to the recipient FSM whose state has been updated to the first-1 state (S4554). In an embodiment, in step S453, based on the list of hospital beds for which the recipient FSM searches, among the remaining tentatively-assigned hospital beds, excluding the existing tentatively-assigned hospital beds, the recipient FSM may be assigned the hospital bed with the earliest expected discharge date (S4554). Then, the remaining waiting patients who have been tentatively-assigned to the remaining tentatively-assigned hospital beds are reassigned to hospital beds with the next expected admission date (S4554). The next expected admission date is later than the expected admission date prior to reassignment and the earliest expected admission date.

For example, in FIG. 4, when patient A receives concession from a waiting patient WP1, the FSM of the waiting patient WP1 may be replaced in a cell with the earliest expected admission date (day 6 in FIG. 4) that is later than the expected admission date before reassignment (day 5 in FIG. 4). The FSMs of the remaining waiting patients (WP2, WP3, . . . ) are also reassigned to the cells associated with the next expected admission date.

In an embodiment, the recipient FSM that transmitted the concession request acceptance signal updates the attribute of the number of concession request acceptance times (S4553). If the recipient FSM never transmits a concession request acceptance signal, it has an initial value (e.g., 0). However, when the recipient FSM transmits a concession request acceptance signal, the attribute value of the number of concession request acceptance times is updated each time the signal is transmitted.

If it is determined that there is no need to accept the concession request based on the urgency of admission (S4552), the recipient FSM maintains its state in the first-2 state (S4555). Additionally, the recipient FSM transmits a concession request rejection signal to the requester FSM (S4555). For example, if the urgency of admission of the requester FSM has the same or lower priority, a concession request rejection signal may be transmitted.

Additionally, in step S4552, it may be determined whether to accept the concession request based on the urgency of admission and the number of acceptance requests. If concessions are requested from the recipient FSM that has accepted too many concession requests, a waiting patient for the recipient FSM may be admitted very late. Although the severity of the recipient FSM is lower, the recipient FSM with an acceptance count greater than or equal to a threshold acceptance count may transmit a concession request rejection signal to the requester FSM, rejecting the concession request and maintaining the hospital bed to which it is assigned (S4555).

Referring to FIG. 6, the smart hospital bed operating method further comprises providing a hospital bed assignment plan table including a hospital bed assignment status (S500).

In an embodiment, the hospital bed assignment plan table may be a plan table indicating the assignment status of waiting patients with the second state, third state, or fourth state.

In another embodiment, the hospital bed assignment plan table may be a plan table indicating the assignment status of waiting patients with the first state, second state, third state, or fourth state.

In addition, the hospital bed operating system and method may perform an operation of reassigning a hospital bed of an inpatient who is already admitted.

In an embodiment, the patient in the hospital bed assignment query input in step S100 may be an inpatient. This is because inpatients may also require rearrangement of hospital beds, such as transfer of department or room, due to changes in their conditions such as worsening/alleviation of the condition.

When a hospital bed assignment query including hospitalization indication for an inpatient is received in step S100, hospital beds matching the conditions for reassignment of the inpatient are searched (S200). When an empty hospital bed is found, the patient is moved to the corresponding hospital bed and rearrangement is completed (S300). If an empty hospital bed is not searched, an FSM corresponding to the hospitalization indication of the inpatient is formed, and rearrangement is performed for the searched hospital beds (S400). The expected discharge date from the hospital bed to be reassigned to the FSM of the inpatient is calculated and used as the expected waiting date of the new waiting patient.

However, in the embodiment, since the inpatient is not admitted to the hospital bed to be rearranged although the inpatient is admitted to the previous hospital bed, the FSM with first to third states is formed and its state is changed to fourth state once the admission to the rearranged hospital bed is completed.

Since hospital bed assignment for inpatients is also performed by treating new hospital beds the same as waiting patients, and is similar to the hospital bed assignment process for waiting patients, detailed explanations are omitted.

Additionally, step S400 may further include calculating the daily discharge probability for some or all sections of the expected length of stay in step 410 (S420). Then, a hospital bed assignment plan table with daily discharge probabilities clearly marked may be provided (S500). The form of providing the hospital bed assignment plan table is described with reference to FIGS. 3 and 4, and detailed description is omitted.

The operations of the smart hospital bed operating system and method according to the embodiments described above may be at least partially implemented as a computer program and recorded on computer-readable recording media. For example, the smart hospital bed operating system and method may be implemented with a program product comprising computer-readable media containing program code, and may be executed by a processor to perform any or all steps, operations, or processes described.

The computer may be a computing device, such as a desktop computer, laptop computer, notebook, smart phone, or the like, or any device that may be integrated. The computer is a device that has one or more alternative and special-purpose processors, memory, storage, and networking components (either wireless or wired). The computer may run an operating system such as, for example, Microsoft's Windows-compatible operating system, Apple's OS X or iOS, a Linux distribution, or Google's Android OS.

The computer-readable recording media include all types of recording and identification devices that store data that can be read by a computer. Examples of computer-readable recording media includes ROM, RAM, CD-ROM, magnetic tape, floppy disk, and optical data storage and identification devices. Additionally, the computer-readable recording media may be distributed across computer systems connected to a network, and computer-readable codes may be stored and executed in a distributed manner. Additionally, functional programs, codes, and code segments for implementing this embodiment may be easily understood by those skilled in the art to which this embodiment belongs.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

### INDUSTRIAL APPLICABILITY

The present invention relates to a hospital bed operating system that optimizes supply according to demand for hospital beds, predicts the supply and demand, and performs balanced management using big data.

1. A smart hospital bed operating method performed by a computing device comprising a processor, the method comprising:
receiving a hospital bed assignment query including a hospitalization indication condition for a waiting patient;
searching for a hospital bed that matches the hospitalization indication condition; and
if a searched list of hospital beds does not include an empty hospital bed, pre-assigning a hospital bed occupied by an inpatient to the waiting patient based on a hospital bed assignment plan table including the expected discharge date of the inpatient.

2. The method of claim 1,
wherein the pre-assigning of the hospital bed occupied by the inpatient to the waiting patient comprises
calculating the expected length of stay of the inpatient by applying inpatient information of the inpatient to a pre-modeled prediction function,
creating a hospital bed assignment plan table including the calculated expected length of stay, and
assigning the hospital bed to the waiting patient based on the hospital bed assignment plan table,
wherein the hospital bed assignment plan table consists of inpatient hospital beds, inpatients occupying the inpatient hospital beds, and days.

3. The method of claim 2,
wherein the expected length of stay of the inpatient is calculated by the prediction function consisting of a dependent variable and a plurality of independent variables, and
is an expected length of stay from a hospitalization indication date to an expected discharge date of the inpatient,
wherein the independent variables comprise one or more of gender, diagnosis code, surgery code, treatment code, or measurement value describing a patient's condition of the waiting patient.

4. The method of claim 3,
wherein coefficients of the independent variables in the prediction function are determined by an optimization algorithm, and the optimization algorithm includes job shop scheduling, Petri net, or CPU scheduling.

5. The method of claim 3,
wherein the hospital bed assignment plan table comprises a plurality of cells, each of which includes an empty cell associated with hospital beds and days and a fill cell associated with hospital beds, inpatients, and days,
wherein the assigning of the hospital bed to the waiting patient based on the hospital bed assignment plan table comprises
creating a finite state machine (FSM) of the waiting patient,
setting a state of the FSM based on the previous history of the waiting patient of the FSM, where if there is no previous history, the state is set to an initial state, and
placing the FSM on the hospital bed assignment plan table according to a code of conduct predefined for each state of the FSM.

6. The method of claim 5,
wherein the initial state is a first-1 state which indicates searching for cells in which the FSM will be placed,
wherein the placing of the FSM on the hospital bed assignment plan table according to the code of conduct predefined for each state of the FSM comprises
placing the FSM whose state is set to the first-1 state in the earliest empty cell among the empty cells in the hospital bed assignment plan table and updating the state of the FSM to a first-2 state, where the earliest empty cell is an empty cell associated with the inpatient hospital bed with the earliest expected admission date in the hospital bed assignment plan table, and
updating the state of the FSM to the second state if there is no event for a first predetermined period of time,
wherein the second state indicates that assignment is completed.

7. The method of claim 6, wherein the placing of the FSM on the hospital bed assignment plan table according to the code of conduct predefined for each state of the FSM further comprises
sending a reservation notification message including the fact that assignment has been completed to a waiting patient of the FSM in the second state,
wherein the reservation notification message includes hospital bed resource information about a hospital bed associated with the cells in which the FSM is placed.

8. The method of claim 6,
wherein if the urgency of admission included in the hospitalization indication condition satisfies a priority condition, the FSM of the waiting patient who satisfies the priority condition comprises an attribute of maximum tolerable waiting time,
wherein the placing of the FSM on the hospital bed assignment plan table according to the code of conduct predefined for each state of the FSM comprises
comparing an expected waiting time for the inpatient hospital bed associated with empty cells in which the FSM in the first-2 state is placed with a preset maximum tolerable waiting time; and
if the comparison results in that the expected waiting time is equal to or shorter than the maximum tolerable waiting time, updating the state of the FSM to a second state.

9. The method of claim 8,
wherein the placing of the FSM on the hospital bed assignment plan table according to the code of conduct predefined for each state of the FSM comprises
if the comparison results in that the maximum tolerable waiting time is longer than the expected waiting time for the tentatively-assigned hospital bed, sending a concession request signal to another FSM, and updating the state of the FSM from the first-2 state to a first-3 state, where the other FSM is a recipient FSM and includes another FSM tentatively assigned to another hospital bed with an expected discharge date that is equal to or earlier than the maximum tolerable waiting time, and
upon receiving a concession request acceptance signal from the recipient FSM, replacing the FSM to cells where the recipient FSM that transmitted the concession request acceptance signal is placed, and updating the replaced FSM to the second state.

10. The method of claim 9, further comprising:
when the recipient FSM has the first-2 state and
receives a concession request signal from a requester FSM, comparing the urgency of admission of the recipient FSM with the urgency of admission of the requester FSM;
when the urgency of admission of the requester FSM is higher than that of the recipient FSM as a result of the comparison, transmitting a concession request acceptance signal to the requester FSM; and
when the urgency of admission of the requester FSM is equal to or lower than that of the recipient FSM, transmitting a concession request rejection signal to the requester FSM.

11. A computer-readable recording medium recording a program for performing the smart hospital bed operating method according to claim 1.

12. A hospital bed operating system using big data, the system comprising:
a hospital bed management processing unit configured to obtain hospital bed resource information, inpatient information, and waiting patient information;
a hospital bed assignment processing unit configured to search for a hospital bed matching a hospitalization indication condition upon receiving a hospital bed assignment query including the hospitalization indication condition, and pre-assign a hospital bed occupied by an inpatient to a waiting patient based on a hospital bed assignment plan table including the inpatient's expected discharge date when the searched list of hospital beds does not include an empty hospital bed; and
a hospital bed schedule prediction processing unit configured to calculate the expected length of stay of the inpatient by applying inpatient information of the inpatient to a pre-modeled prediction function.

13. The system of claim 12,
further comprising a database storing the hospital bed resource information, the inpatient information, and the waiting patient information.

14. The system of claim 12,
further comprising a user interface provision unit that provides the hospital bed assignment plan table to a user.
